(19) United States
(12) Patent Application Publication
(10) Pub. No.: US 2024/0065716 A1
(43) Pub. Date: Feb. 29, 2024

(54) SEALER - DIVIDER - DISSECTOR AND RELATED METHODS

(71) Applicant: BOLDER SURGICAL, LLC, Marlborough, MA (US)

(72) Inventors: Nathan Steketee, Marlborough, MA (US); Timothy Crowley, Marlborough, MA (US); Christopher Deborski, Marlborough, MA (US); Russell Hempstead, Marlborough, MA (US); Ryan Smith, Marlborough, MA (US); John Twomey, Marlborough, MA (US)

(73) Assignee: BOLDER SURGICAL, LLC, Marlborough, MA (US)

(21) Appl. No.: 18/494,775

(22) Filed: Oct. 26, 2023

Related U.S. Application Data
(63) Continuation of application No. PCT/US2022/026779, filed on Apr. 28, 2022.
(60) Provisional application No. 63/180,782, filed on Apr. 28, 2021.

(51) Int. Cl. A61B 17/29

(52) U.S. Cl. CPC A61B 17/29; A61B 2017/2919; A61B 2017/2947

(57) ABSTRACT

A surgical device includes a pair of jaws for manipulating tissue disposed therebetween; a housing and a hemostat style gripping mechanism; an elongated shaft positioned between the housing and the pair of jaws and defining a longitudinal axis; and a pull tube at least partially disposed within the elongated shaft. The hemostat style gripping mechanism comprises a linkage system configured to move the jaws between an open position and an approximated position, the linkage system comprises a first shank having distal end rotatably coupled to the housing at a first fixed pivot point and a proximal end coupled to a first finger grip, a second shank having a distal end rotatably coupled to the housing at a second fixed pivot point and a proximal end coupled to a second finger grip, and a slider link operatively coupled to the first shank and the second shank.

### CROSS-REFERENCE TO RELATED APPLICATIONS

The present application is a continuation of International Application No. PCT/US2022/026779, filed Apr. 28, 2022, which claims the benefit under 35 U.S.C. 119 to U.S. Provisional Application No. 63/180,782, filed Apr. 28, 2021, the contents of all of which are hereby incorporated herein by reference in their entirety into the present application.

### FIELD

This invention is related to surgical instruments. Specifically, but not intended to limit the invention, embodiments of the invention are related to surgical instruments for sealing tissue during surgical procedures.

### BACKGROUND

Surgeons often perform procedures in small or compact spaces of the body, such as the head/neck area or other parts of the body, as for example, thyroid resection, parathyroid resection, and/or other resections. Small or compact areas in the body are tightly packed with structures such as nerves, arteries, thyroid, esophagus, muscles, and/or other narrow spaces that inhibit surgeon's ability to maneuver around during surgical procedures (e.g., open, general, or laparoscopic).

Currently available medical instruments are used in surgical procedures that may cause trauma in patients. By way of illustration, FIG. 1 depicts a hemostat style sealer-divider used in open surgical procedures. However, the hemostat style sealer-divider of FIG. 1 is not suited for laparoscopic procedures due to the large scissor-style arms. Additionally, the hemostat style sealer-divider is also difficult to use in certain open procedures, such as in tight body spaces that further limits visibility for the surgeon. Moreover, when using the device shown in FIG. 1, the maximum opening of the jaws is limited by the size of the surgeon's hands.

By way of further illustration, surgeons may use a sealer-divider having a pistol grip, as shown in FIG. 2. Although the sealer-divider having a pistol grip allows for laparoscopic procedures, the pistol grip may be unstable, particularly when surgeons close or open the end effector during surgical procedures. For example, instability may be caused when the pistol grip device is used during open procedures due to the need for greater movement of hands/fingers to reach actuators, which in turn causes surgeons to inadvertently contact nerves. Many of the pistol grip devices also include a ratchet mechanism that engages during jaw closure (intended to optimize pressure on tissue during tissue sealing); such that, after each seal, the surgeon must ‘click’ the handle closed to disengage the ratchet mechanism and allow the jaws to open, introducing further instability during surgical procedures. Such instability increases patient trauma, particularly when working in tight spaces (e.g., head and neck area).

Some of the patient trauma is caused when surgical instruments inadvertently strike nerves and/or when electrical energy, such as that applied during a tissue sealing procedure, spreads too far beyond the intended target tissue. To minimize patient trauma, it is desirable to minimize collateral thermal damage, as well as unnecessary contact with sensitive tissues during surgical procedures; achieving this, however, is a challenge when working in tight spaces, such as the head and neck area, particularly when using currently-available devices.

Embodiments described herein overcome these and/or other problems by providing a multi-functional device that minimizes instrument instability during surgical procedures, improves surgeon control, increases surgeon visibility of the distal end of the device, reduces device size while providing a large jaw opening, and/or reduces the time needed for surgical procedures, some or all of which may reduce patient trauma and improve surgical outcomes. Another advantage of embodiments described herein is the ability to wide open the jaws of the instrument, which is beneficial during tissue dissection, in a manner that is independent of the size of a surgeon's hand.

### SUMMARY

In one embodiment, a surgical device comprises a distal portion having a pair of jaws configured to move between an open position and an approximated position for manipulating tissue disposed therebetween; a proximal portion having a housing and a hemostat style gripping mechanism, the hemostat style gripping mechanism including a first finger grip and a second finger grip; an elongated shaft positioned between the proximal portion and the distal portion, the elongated shaft defining a longitudinal axis; and a pull tube at least partially disposed within the elongated shaft. The hemostat style gripping mechanism of the surgical device comprises a linkage system configured for effectuating movement of the pair of jaws between the open position and the approximated position, the linkage system comprises a first shank having distal end rotatably coupled to the housing at a first fixed pivot point and a proximal end coupled to the first finger grip, a second shank having a distal end rotatably coupled to the housing at a second fixed pivot point and a proximal end coupled to the second finger grip, and a slider link operatively coupled to the first shank and the second shank. The pull tube of the surgical device comprises a proximal end coupled to the slider link and a distal end coupled to the pair of jaws, the pull tube configured to move between a first position and a second position proximal of the first position in response to manipulation of the first shank and/or the second shank, whereby the pair of jaws are moved between the open position and the approximated position.

In some embodiments, the linkage system is a 7-bar linkage system comprising the first shank, second shank, housing, and slider link. The linkage system further comprises a first lever link rotatably coupled to the first shank at each of a first floating pivot point and the slider link, a second lever link rotatably coupled to the second shank at a second floating pivot point and the slider link, and a slide track link affixed to or defined by the housing and configured to limit the slider link to longitudinal movement relative to the housing.

In some embodiments, the first and second shanks, and the second lever links, and the first and second floating links are collectively configured to vary a mechanical advantage between the open position and the approximated position. In some embodiments, the mechanical advantage occurs when the first and second shanks move from the open position to the approximated position during a stroke, the pair of jaws travel a greater distance during a first half of the stroke than during a second half of the stroke. Further, the mechanical advantage occurs where a compressive force needed to move the first and second finger grips closer together decreases as the pair of jaws approaches the approximated position.

In some embodiments, the first and second floating pivot points are configured move outwardly from the longitudinal axis defined by the elongated shaft when the pull tube moves distally. Conversely, the first and second floating pivot points are configured move inwardly from the longitudinal axis defined by the elongated shaft when the pull tube moves proximally.

In these embodiments, the linkage system is configured to prevent locking of the pair of jaws in the approximated position. Optionally, the first and second floating pivot points are configured to lock the pair of jaws in the approximated position when the first shank and the first lever link form an angle of approximately 180 degrees therebetween.

In some embodiments, the slider link comprises a spring housing that houses a load-limiting spring, and wherein the load-limiting spring is configured to limit a pull force on the pull tube when the pair of jaws are in the approximated position. The load-limiting spring is configured to disengage from the pull tube during a transition of the pair of jaws from the approximated position to the open position.

The surgical device further comprises a tissue sealing system having an electrode actuator, a first electrode disposed on a first jaw of the pair of jaws, and a second electrode disposed on a second jaw of the pair of jaws. The first and second electrodes are configured to seal the tissue disposed between the pair of jaws in response to a proximal movement of either one of the electrode actuators, wherein the electrode actuators move in response to a force applied to either one of the electrode actuators in a direction parallel to the longitudinal axis defined by the elongated shaft. The electrode actuators are disposed along and laterally offset from the longitudinal axis defined by the elongated shaft.

In some embodiments, the linkage system is configured to vary a mechanical advantage between the open position and the approximated position. In some embodiments, the mechanical advantage occurs when the first and second shanks of the linkage system move from the open position to the approximated position during a stroke, the pair of jaws travel a greater distance during a first half of the stroke than during a second half of the stroke. In this embodiment, the mechanical advantage decreases throughout the stroke. In some embodiments, the mechanical advantage occurs where a compressive force needed to move the first and second finger grips decreases as the pair of jaws approaches the approximated position.

In another embodiment, a surgical device configured to manipulate tissue is disclosed. The surgical device comprises a handle operably coupled to an end effector by a linkage system and an elongated shaft, the handle comprising a first shank and a second shank, the end effector comprising opposing jaws selectively movable between an open position and an approximated position when the handle is actuated. The linkage system is configured so that a stroke of the handle moves the opposing jaws to an approximated position to engage tissue disposed therebetween by moving the first and second shanks closer together, and wherein a mechanical advantage varies during the stroke whereby the opposing jaws travel a greater distance during a first half of the stroke than during a second half of the stroke.

In some embodiments, the linkage system is configured such that moving the first and second shanks causes the first shank to rotate about a first fixed pivot of the linkage system and causes the second shank to rotate about a second fixed pivot the linkage system.

Optionally, the first and second fixed pivots are laterally offset from a longitudinal axis defined by the elongated shaft.

In some embodiments, the surgical device further comprises one or more electrode actuators each having a contact surface for a user's finger, wherein at least one of the electrode actuators is disposed along and laterally offset from the longitudinal axis defined by the elongated shaft. The contact surface of the at least one of the actuators is radially offset from the longitudinal axis by a first distance, and the fixed pivot points are radially offset from the longitudinal axis by a second distance greater than the first distance.

In some embodiments, the surgical device further comprises a protrusion coupled to the slider link, wherein at least a portion of the protrusion is slidably disposed within a guide member, and wherein the protrusion and the guide member are configured to prevent rotation of the slider link.

In yet another embodiment, a method of operating a surgical device to manipulate tissue is disclosed. The surgical device use by method of operating comprises a handle operably coupled to an end effector by a linkage system and an elongated shaft, the handle comprising a first shank and a second shank and the end effector comprising opposing jaws and selectively movable between an open position and an approximated position using the handle. The method comprises moving the opposing jaws to an approximated position to engage tissue disposed therebetween by moving the first and second shanks closer together while varying a mechanical advantage as the opposing jaws move to the approximated position. Moving the first shank and the second shank comprises rotating the first shank about a first fixed pivot and the second shank about a second fixed pivot. The first fixed pivot and the second fixed pivot are laterally offset from a longitudinal axis defined by the elongated shaft.

In some embodiments of the method of operating the surgical device, the device further comprises a load-limiting spring; and the method further comprises engaging the load-limiting spring when the pair of jaws are in the approximated position, and disengaging the load-limiting spring when the pair of jaws approaches the open position. The method may further comprise moving the pair of jaws between the open position and the approximated position without locking the pair of jaws in the approximated position.

In other embodiment, a method of operating an instrument having a hemostat-style gripping mechanism mechanically coupled to a pair of jaws to open and close the pair of jaws, the pair of jaws including electrodes electrically coupled to an actuator disposed on a proximal portion of the instrument and configured to selectively actuate the electrodes is disclosed. The method comprises actuating the electrodes by applying a compressive force to the actuator in a direction parallel to a longitudinal axis of the instrument. In the embodiment where the actuator has an outer surface radially offset from the longitudinal axis by a distance, the method further comprises rotating a first shank and a second shank about respective pivot points, where the pivot points are radially offset from the longitudinal axis by a first distance, and where the outer surface of the actuator is radially offset from the longitudinal axis by a second distance, and wherein the second distance is greater than the first distance.

### BRIEF DESCRIPTION ON THE DRAWINGS

The drawings illustrate the design and utility of embodiments, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only exemplary embodiments and are not therefore to be considered limiting in the scope of the claims.

FIG. 1 is a perspective view of an exemplary device (Prior Art);

FIG. 2 is a perspective view of another exemplary device (Prior Art)

FIG. 3 is a perspective view of a sealer-divider-dissector, according to the embodiments of the invention;

FIG. 4 is a first side view of the device of FIG. 3;

FIG. 5 is a second side view of the device of FIG. 3;

FIG. 6 is a perspective view of some components of the device of FIG. 3;

FIG. 7 is a first side section view of some components of FIG. 3, having the schematic illustration of the linkage components of FIG. 8 superimposed;

FIG. 8 is a first side schematic illustration of the linkage components of FIG. 6;

FIG. 9 is a first side view of the device of FIG. 3 with some components removed for clarity;

FIG. 10 is a second side view of the device of FIG. 3 with some components removed for clarity;

FIG. 11 is a perspective exploded view of the device of FIG. 3 with some components removed for clarity;

FIG. 12 is a first side view of some components of the device of FIG. 3, with some features removed for clarity;

FIG. 13 is a perspective view of a distal end of the device of FIG. 3 grasping tissue;

FIG. 14 is a side view of the distal end of the device of FIG. 3 grasping tissue, with some components removed for clarity;

FIGS. 15A-15E illustrate schematic sketches of differences between the devices of FIGS. 1-3; and

FIG. 16 is a flow chart of an exemplary method of use of the device of FIG. 3.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skilled in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Various embodiments are described hereinafter with reference to the figures. The figures are not necessarily drawn to scale, the relative scale of select elements may have been exaggerated for clarity, and elements of similar structures or functions are represented by like reference numerals throughout the figures. It should also be understood that the figures are only intended to facilitate the description of the embodiments and are not intended as an exhaustive description of the invention or as a limitation on the scope of the invention, which is defined only by the appended claims and their equivalents. In addition, an illustrated embodiment needs not have all the aspects or advantages shown. An aspect or an advantage described in conjunction with a particular embodiment is not necessarily limited to that embodiment and can be practiced in any other embodiments even if not so illustrated.

Those skilled in the art will recognize that the term “tissue dissection” as used herein refers to the practice of piercing tissue while the jaws are closed or close to one another, then separating the tissue by opening the device jaws or moving the jaws away from each other. In contrast, the term “tissue dividing” is used herein to refer to the practice of cutting tissue clamped between the jaws of the device.

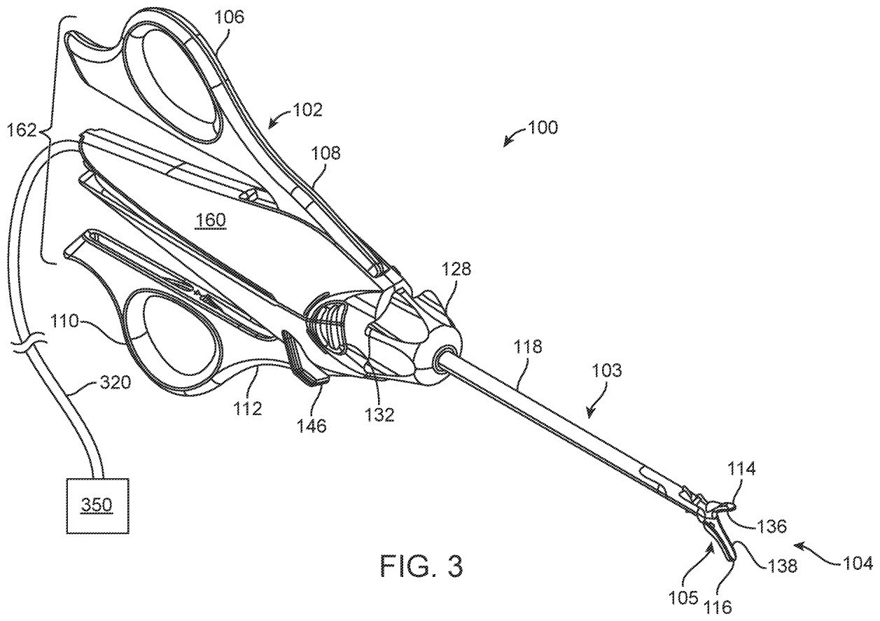

FIGS. 3-5 illustrate perspective, first side, and second side views, respectively, of a sealer-divider-dissector device 100, according to embodiments of the invention. The device 100 comprises a proximal portion 102, a distal portion 104, and a body portion 103 disposed therebetween. The proximal portion 102 is configured to allow a user (e.g., surgeon, clinician) to grip, handle and actuate the device 100 using a single hand. The proximal portion 102 comprises an actuator, such as a handle. The distal portion 104 is configured to grasp, hold, and/or manipulate tissue. The distal portion 104 is further configured to seal, divide, and/or dissect tissue when the device 100 is actuated, as it will be described in further detail below. The body portion 103 comprises an elongated shaft 118 configured to transfer surgeon's actuation (e.g., translation and/or rotation) of the proximal portion 102 to the distal portion 104 of the device 100. The elongated shaft 118 and/or distal portion 104 may be rotatable relative to the proximal portion 102 of the device 100.

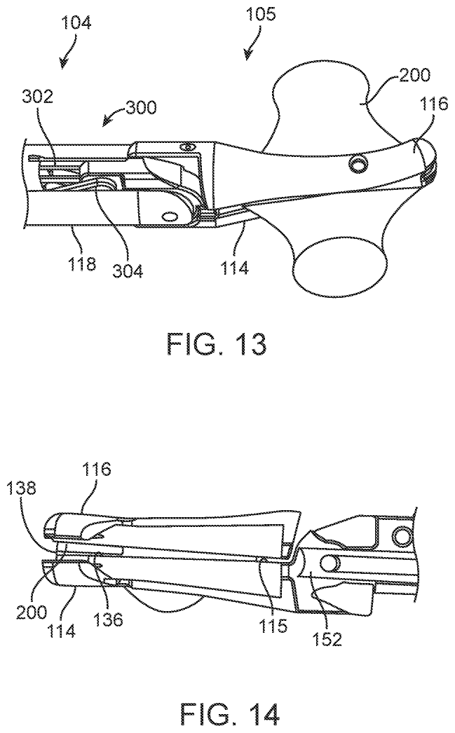

The distal portion 104 of the device 100 comprises an end effector 105 having a pair of jaws (i.e., first jaw 114 and second jaw 116). The jaws 114 and 116 are configured to move between an open position (as shown in FIGS. 3-5) and an approximated position (as shown in FIG. 13 and FIG. 14). An approximated position is defined as the position where the jaws 114 and 116 are closed while grasping, holding and/or restraining tissue disposed therebetween. Those skilled in the art will recognize that the approximated position is dictated by the thickness of the tissue, and that the thickness of the tissue may change during a surgical procedure, such as during tissue sealing and desiccation. It should be appreciated that one or both surfaces (e.g., a first electrode 136 and a second electrode 138) of the jaws 114 and 116 may comprise one or more non-conductive stop members 115 (as shown in FIG. 14) preventing direct contact of the surfaces of the jaws 114 and 116, avoiding electrical short, in a manner known to those skilled in the art.

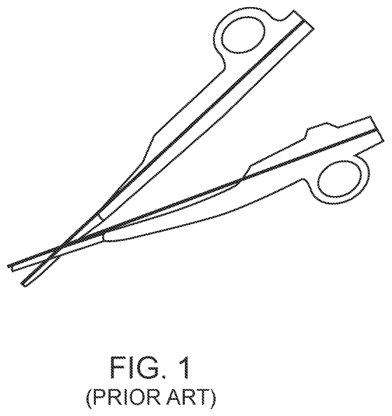

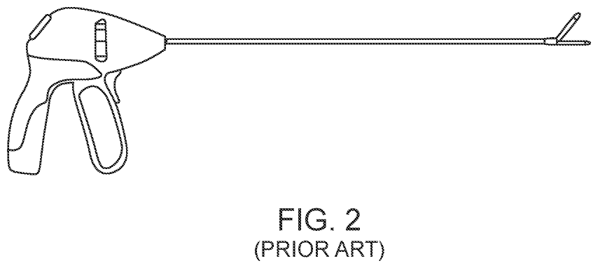

The proximal portion 102 of the device 100 comprises a housing 160 and a hemostat style gripping mechanism, handle or actuator 162. The housing 160 and/or hemostat style gripping mechanism/actuator 162 may be formed of one or more components. A hemostat style gripping mechanism is defined as a mechanism configured for a user's grip in a fashion similar to the hemostat device of FIG. 1 (i.e., distinct from the pistol grip device of FIG. 2).

As shown in FIG. 3, an electrical wire or wires 320 extend from a power source 350, such as a radio frequency generator, through the housing 160 to the end effector 105; the electrical wires 320 are configured to deliver energy to the end effector 105. In an alternative embodiment, the electrical wires 320 may extend from the actuator 162 to the end effector 105. In some embodiments, the connection of the device 100 to the power source 350 is wireless (not shown). The energy delivered may be radio-frequency (RF) energy or any other suitable energy for sealing, dividing and cutting tissue.

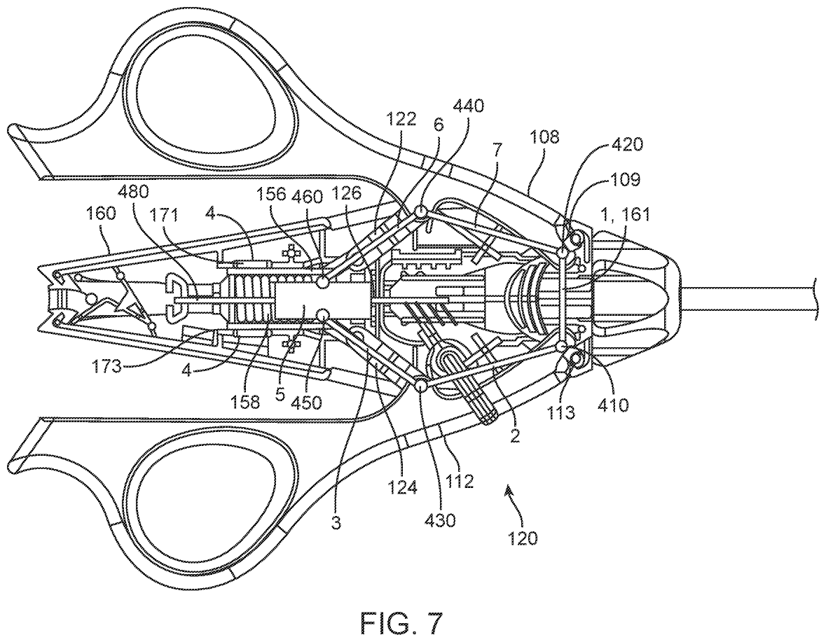

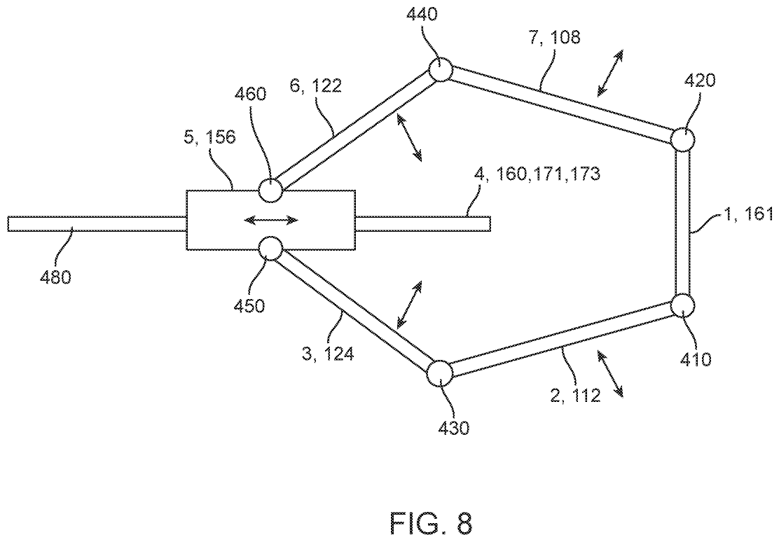

As shown in FIGS. 3-5, the proximal portion 102 of the device 100 further comprises a first shank 112 and a second shank 108 and a linkage system 120 (FIG. 7 and FIG. 8) to actuate the jaws 114 and 116 into open and/or approximated (closed) positions. The first shank 112 is coupled to a first finger grip 110, and the second shank 108 is coupled to a second finger grip 106. Although the finger grips 110 and 106 are illustrated as rings, those skilled in the art will recognize that the finger grips 110 and 106 may comprise any suitable configuration that allows a reliable grasp on the device 100; for example, a concave/wave shape, indentation and/or recess in one or both shanks 112 and 108.

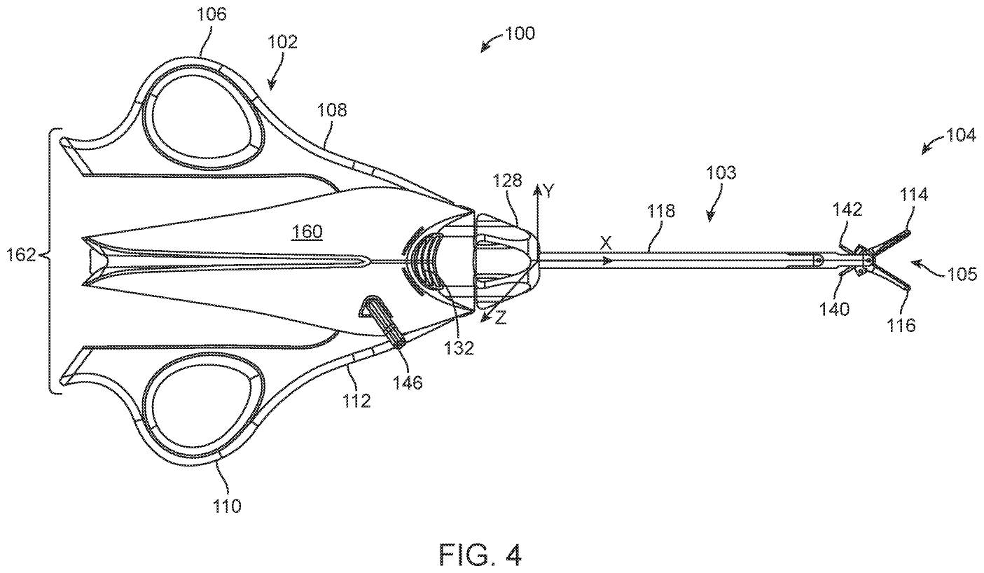

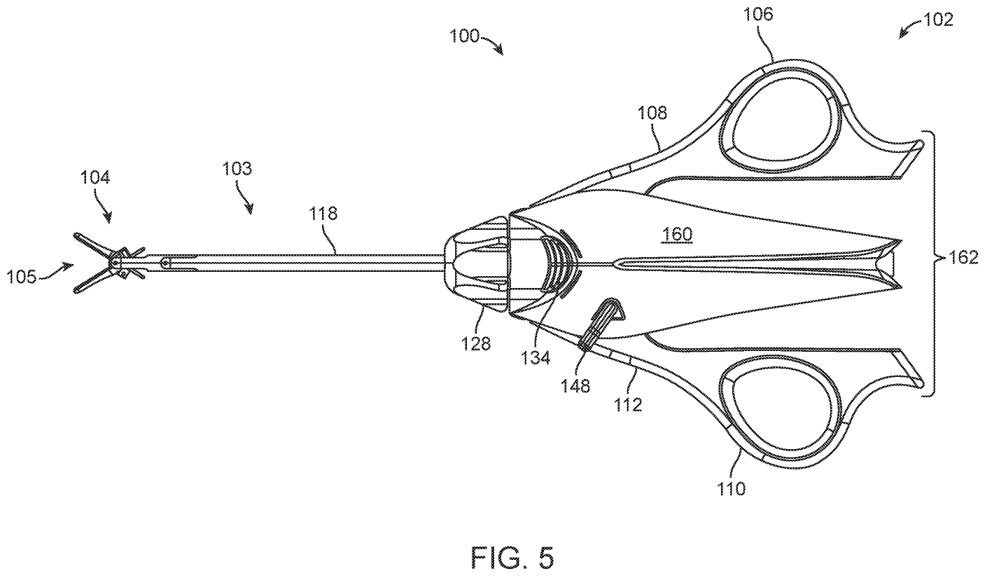

The proximal portion 102 of the device 100 further comprises a finger wheel 128 coupled to the elongated shaft 118. The finger wheel 128 is configured to rotate the elongated shaft 118 and/or jaws 114, 116 relative to the proximal portion 102 (e.g., housing 160) of the device 100, in response to actuation of the finger wheel 128. In some embodiments, the proximal portion 102 of the device comprises an electrode actuator 132 and a knife actuator 146 on one side (as shown in FIG. 3 and FIG. 4), and an electrode actuator 134 and a knife actuator 148 in another side (as shown in FIG. 5); the electrode and knife actuators will be described in further detail below.

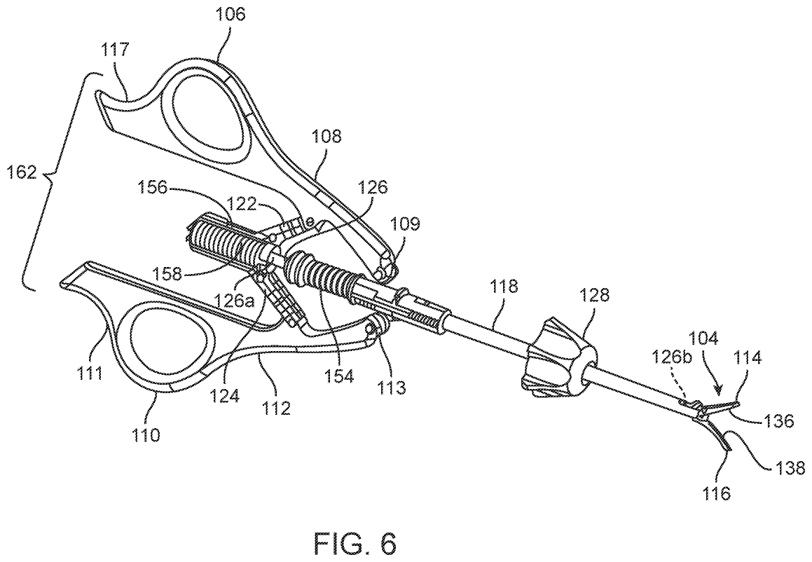

FIG. 6 illustrates a perspective view of the device 100 with some features removed for clarity. Specifically, FIG. 6 illustrates the hemostat style gripping mechanism 162 of the proximal portion 102 of the device 100 coupled to the elongated shaft 118 of the body portion 103 and the end effector 105 of the distal portion 104. The hemostat style gripping mechanism 162 comprises and/or is coupled to a linkage system 120 for effectuating movement of the pair of jaws 114, 116 between the open position and the approximated position.

FIG. 7 and FIG. 8 illustrate details of the linkage system 120 of the device 100. Particularly, FIG. 8 is a schematic illustration of the linkage system (120); the schematic illustration of FIG. 8 is superimposed over the device 100 depicted in FIG. 7. As shown in FIGS. 6-8, the linkage system 120 comprises the first shank 112 and the second shank 108. Each shank 112 and 108 have respective distal ends 113 and 109. The distal ends 113, 109 of the shanks 112,108 are rotatably coupled to the housing 160 (FIG. 7). The finger grip 106 is disposed at or near a proximal end 117 of the shank 108, and the finger grip 110 is disposed at or near a proximal end 111 of the shank 112 (FIG. 6). The linkage system 120 further comprises a first lever link 124 and a second lever link 122. The first lever link 124 is rotatably coupled to the first shank 112 and a cartridge spring housing 156, while the second lever link 122 is rotatably coupled to the second shank 108 and the cartridge spring housing 156. The cartridge spring housing 156 may be limited to longitudinal movement relative to the housing 160. The cartridge spring housing 156 comprises a load-limiting spring 158. Rotational movement of the shanks 112, 108 about their respective distal ends 113 and 109 is configured to transfer to the cartridge spring housing 156 and to a tubular member 126, such that, the tubular member 126 effectuates rotation of the jaws 114, 116. In some embodiments, the tubular member 126 is a hollow pull tube or a rod. The linkage system 120 may be operatively coupled to a jaw linkage system 300 (FIG. 13) or to the jaws 114, 116. The linkage system 120 is configured to move the jaws 114, 116 in response to manipulation of the shanks 112, 108.

As shown in FIGS. 6-8, the linkage system 120 comprises a 7-bar linkage system configured to convert rotating movement of the shanks 112, 108 into longitudinal movement of the pull tube 126. To facilitate the disclosure of the linkage system 120, the links will be described in the order shown in FIG. 8 from right to left (instead of numerical order). The linkage system 120 comprises a base link 1 fixedly coupled to or defined by the housing 160. The base link 1 may be a feature 161 in or is affixed to the housing 160. The base link 1 comprises a first fixed pivot point 410 and a second fixed pivot point 420; where each of the pivot points 410, 420 are coupled to the housing (i.e., no rotation or translation of the pivot points). The linkage system 120 further comprises a link 2 and a link 7. The link 2 may be the first shank 112, and the link 7 may be the second shank 108. The linkage system 120 comprises a link 3 and a link 6. The link 3 may be the first lever link 124, and the link 6 may be the second lever link 122. The first lever link 124 is rotatably coupled to the first shank 112 at a first floating pivot point 430 and the second lever link 122 is rotatably coupled to the second shank 108 at a second floating pivot point 440. The floating pivot points 430, 440 are configured to allow movement of the links 3 and 6, relative to their respective coupling links 2 and 7, as shown by arrows in FIG. 8, in response to the user's movement of shanks 112 and 108. Such as, the first floating pivot point (430) and the second floating pivot point (440) are configured to move outwardly from the longitudinal axis of the device 100, when the pull tube (126) moves distally, and further configured to move inwardly from the longitudinal axis of the device 100, when the pull tube (126) moves proximately.

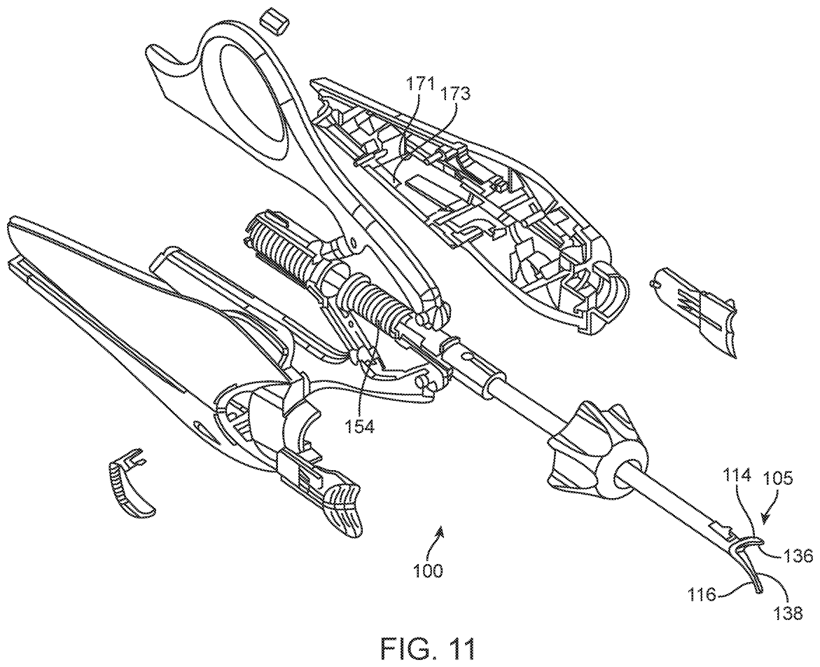

The linkage system 120 further comprises a slider link 5 coupled to links 3 and 6. The slider link 5 may be the cartridge spring housing 156. The cartridge spring housing 156 is coupled to first lever link 124 and second lever link 122, via respective pivot points 450 and 460. The cartridge spring housing 156 is configured to translate, as shown by arrow in FIG. 8, in response to movement of the lever links, 124, 122. The linkage system 120 comprises a slide track link 4. The slide track link 4 may be a feature in or is affixed to the housing 160. The slide track link 4 may include one or more flange surfaces 171, 173 (FIGS. 6-8 and 11-12) configured to permit longitudinal movement of the slider link 5 or cartridge spring housing 156 relative to the housing 160.

Optionally, the cartridge spring housing 156 may comprise a protrusion (not shown), where at least a portion of the protrusion is configured to slidably engage or be slidably disposed within a guide member 480. The guide member 480 comprises a complementary groove, track or any other suitable guiding mechanism (not shown) configured to receive and slidably engage the protrusion of the cartridge spring housing 156. The guide member 480 and the protrusion are configured to guide and thereby limit movement of the cartridge spring housing 156 to two degrees of freedom (i.e., allowing translation along the X-axis, and preventing rotation of the cartridge spring housing 156 within the housing 160 of the device 100). As shown in FIG. 7 and in the schematic illustration of the linkage system 120 of FIG. 8, the guide member 480 is slidably coupled to the cartridge spring housing 156 and shown on the opposite side of the slide track link 4. It should be appreciated that the guide member 480 may be laterally disposed to the cartridge spring housing 156 and/or from a clockwise rotation of the X-Z planes (FIG. 4) of the device 100, the guide member 480 may be disposed underneath or below to the cartridge spring housing 156, or any other suitable position that restricts movement of the cartridge spring housing to translation along the x-axis.

It should be appreciated that the length of the links may vary to change the leverage of the linkage system 120 and increase the mechanical advantage of the device 100. The linkage system 120 is configured to provide a controlled motion of the jaws 114, 116.

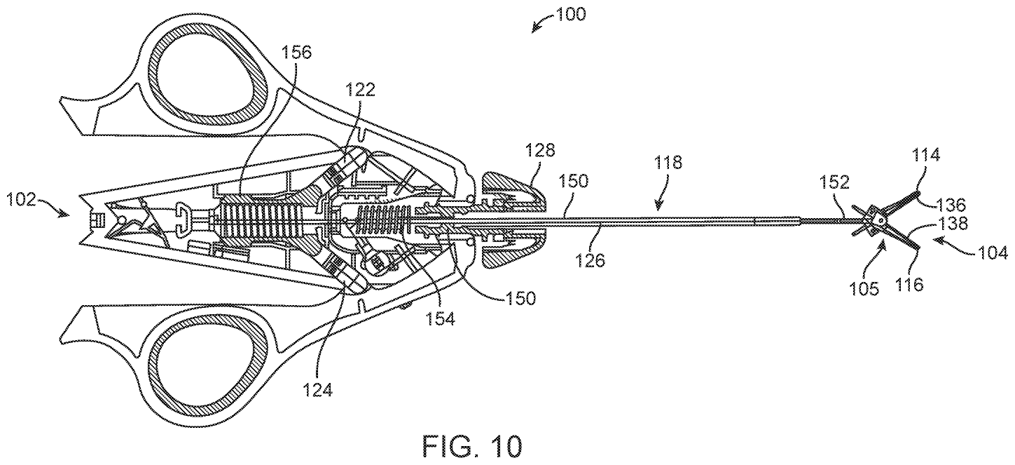

To operate the linkage system 120, a user may move the shanks 112, 108 near to each other (e.g., close or approximate position) causing the cartridge spring housing 156 to move proximally (e.g., in a proximal direction within and/or with respect to the housing 160). Additionally, the user may move the shanks 112, 108 farther away from each other (e.g., open position) causing the cartridge spring housing 156 to move distally (e.g., in a distal direction within and/or with respect to the housing 160). The cartridge spring housing 156 is coupled to the pull tube 126 (FIG. 7 and FIG. 10) such that proximal motion of the cartridge spring housing 156 causes proximal translation of the pull tube 126, which in turn causes the approximated position (e.g., closing) of the jaws 114, 116. Conversely, the distal motion of the cartridge spring housing 156 causes distal translation of the pull tube 126 which in turn causes opening of the jaws 114, 116. Therefore, moving the shanks 112, 108 farther away from each other (open position), opens the jaws 114, 116. As shown in FIG. 6, the pull tube 126 comprises a proximal end 126a coupled to the cartridge spring housing 156 and a distal end 126b coupled to the jaws 114, 116.

In some embodiments, the linkage system 120 is configured to provide an optimized mechanical advantage at the end of a closing stroke (approximated position), such as when the shanks 112, 108 are moved near each other (e.g., towards the housing 160). For example, the mechanical advantage of the device 100 occurs when the first and second shanks 112, 108 of the linkage system (120) move from the open position to the approximated position during a stroke, the pair of jaws 114, 116 travels a greater distance during a first half of the stroke than during a second half of the stroke. Notably, when the jaws 114, 116 grasp, hold, and/or retain tissue 200, the jaws 114, 116 are considered to be in an approximated position (FIG. 13). The approximate position is determined by the thickness of the tissue 200 between the jaws 114, 116. When the jaws 114, 116 are in the approximated position, the pull tube 126 is prevented from further moving proximally, such that further approximation of the shanks 112, 108 causes the cartridge spring housing 156 to engage the load-limiting spring 158 in a manner known to those skilled in the art. As tissue 200 between the jaws 114, 116 is dehydrated, desiccated or fused, the tissue 200 tends to become thinner, which redefines the approximated position of the jaws 114, 116 and allows the pull tube 126 and cartridge spring housing 156 to move proximally during the process. In some embodiments, the load-limiting spring 158 may be configured to limit a pull force on the pull tube 126 to about 40 pounds (18.14 kilograms force) or less, or about 35 pounds (15.88 kilograms force) or less, or about 30 pounds (13.6 kilograms force) or less.

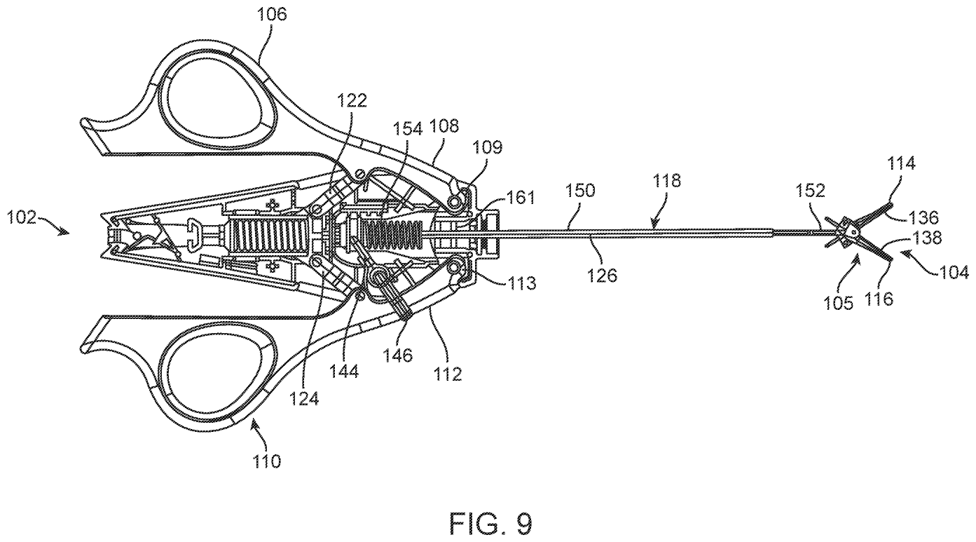

Those skilled in the art will recognize that at least a portion of the pull tube 126 may be positioned inside the elongated shaft 118 (FIG. 9 and FIG. 10). The pull tube 126 may be operatively coupled to the linkage system 120 and/or the cartridge spring housing 156. The pull tube 126 is configured to slide or move relative to the elongated shaft 118, between a first position and a second position. The first position of the pull tube 126 is in response to movement of shanks 112 and/or 108 (e.g., approximate or closed position) and the second position of pull tube 126 is in response to movement of shank 112 and/or 108 (e.g., open position). Movement of the pull tube 126 may engage the jaw linkage system 300 (FIG. 13) of the end effector 105 at the distal portion 104 of the device 100, where the pair of jaws 114, 116 are moved between the open position and the approximated position. The jaw linkage system 300 comprises links 302 and 304, which are similar to jaw linkage systems known to those skilled in the art for operating the jaws 114, 116. Approximating the shanks 112, 108 approximates the jaws 114, 116. Opening the shanks 112, 108 opens the jaws 114, 116. Therefore, both jaws 114, 116 move in response to movement of the shanks 112, 108, as shown in the embodiments disclosed herein. In an alternative embodiment, only one of the jaws (either 112 or 108) move relative to the other jaw (not shown).

FIGS. 9-12 illustrate perspective, detailed and exploded views of the device 100, according to embodiments of the invention. The end effector 105 of the device 100 comprises a cutting mechanism or knife 152 slidably dispose between the jaws 114, 116. In some embodiments, at least a portion of the knife 152 is disposed within and translatable relative to the elongated shaft 118. The knife 152 is translatable between a first position (e.g., retracted, away from the jaws or in proximal direction, shown in FIGS. 9-10 and FIG. 14) and a second position (e.g., active, between the jaws or in distal direction, not shown). The knife 152 moves to the second position in response to user manipulation of either of the knife actuators 146, 148. The knife actuators 146, 148 are configured to move the knife 152 distally to the second position in response to a proximal motion of the knife actuators 146, 148. The second position (e.g., active distal direction) of knife 152 is configured to cut, sever and/or separate the tissue 200 grasped, held and/or restrained between the jaws 114, 116. The device 100 may further include a biasing mechanism, such as a knife spring 154, to bias the knife 152 toward the first position (e.g., retracted or proximal direction). A knife tube 150 may be coupled to or be unitarily formed with the knife 152. The knife tube allows movement of the knife 152 by the knife actuators 146, 148. The knife tube 150 is configured to allow movement of the knife 152 regardless of a rotation and/or orientation of the proximal end 102 of the device 100 relative to the housing 160 or vice versa.

In some embodiments, the pair of jaws 114, 116 are shaped to dissect tissue disposed between the pair of jaws in response to an opening motion of the first shank 112 and the second shank 108 (not shown).

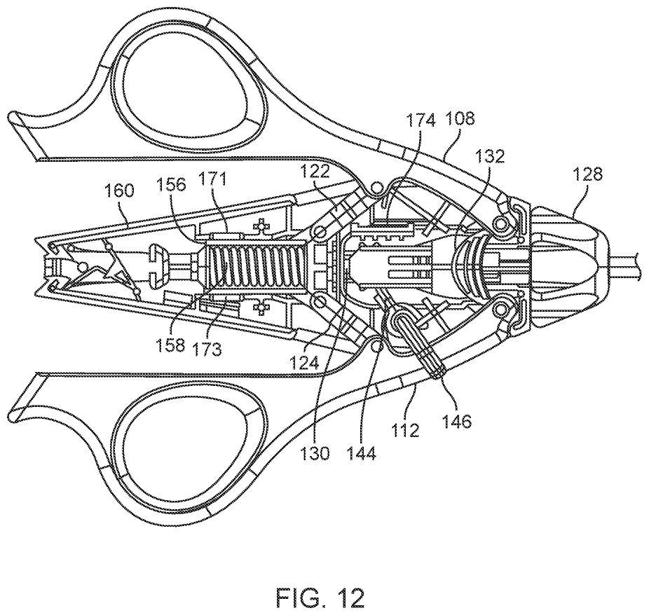

The end effector 105 of the device 100 comprises a first electrode 136 disposed in the first jaw 114 and a second electrode 138 disposed in the second jaw 116 (FIG. 6 and FIGS. 9-11). The device 100 further comprises a first electrode actuator 132 (FIG. 4 and FIG. 12), and a second electrode actuator 134 (FIG. 5). The first and second electrodes 136, 138 are configured to deliver sealing energy to the tissue 200 disposed, grasped, held and/or restrained between the jaws 114, 116 (FIG. 13) in response to movement (e.g., in a proximal direction) of either of the electrode actuators 132, 134. The electrode actuators 132, 134 may be or include a switch 130 (FIG. 12) for electrically activating the electrodes 136, 138. Those skilled in the art will recognize that the conductive wires 320 connect the electrodes 136, 138 to the power source 350 such as a generator (FIG. 3). The generator is described in commonly-owned U.S. Pat. No. 10,342,599 issued on Jul. 9, 2019, which is hereby incorporated by reference in its entirety. Those skilled in the art will further recognize that the sealing system may include features disclosed in commonly-owned U.S. Pat. No. 9,144,455 issued on Sep. 29, 2015, which is hereby incorporated by reference in its entirety. The teachings of commonly-owned U.S. Pat. No. 10,765,471 issued on Sep. 8, 2020 are also incorporated by reference in its entirety.

The sealing system may be configured as a low-power sealing system having a maximum power of 60 Watts and a maximum power of 2.5 Amperes to seal tissue disposed, grasped, held and/or restrained between the jaws 114, 116, and may be configured to seal tissue in 1 second or less.

In some embodiments, the knife actuators 146, 148 are positioned proximal of the electrode actuators 132, 134, which may improve device stability, due to the position of the user's hand during treatment of a patient. In some embodiments, the knife actuators 146, 148 are coupled to a knife trigger yoke 144 and/or knife tube 150 to effectuate movement of the knife 152 (FIG. 9).

In some embodiments, the electrode actuators 132, 134 are disposed along and laterally offset from an axis defined by the elongated shaft 118 (e.g., X axis in FIG. 4). Positioning the electrode actuator 132, 134 on a plane that intersects this axis minimizes movement of the distal portion 104 of the device 100 during surgical procedures, improving device stability. For example, as shown in the coordinate systems depicted in FIG. 4, the electrode actuator 132 is configured to move parallel to a longitudinal X axis defined by the elongated shaft 118, thus increasing the stability of the device 100 during use. Positioning the electrode actuators 132, 134 in close proximity to the X axis of the device 100 (FIG. 4) is achieved by the use of the previously described 7-bar linkage system 120 (FIGS. 6-8). The linkage system 120 is configured to allowed suitable room or space in the center of the housing 160 such as to place the electrode actuators 132, 134 (FIG. 12). In prior art devices, the electrode actuators are substantially offset from the X axis of the device and/or are actuated by applying pressure perpendicular to the device shaft, creating undesirable movement of the distal end of the device when the electrodes are actuated by the user. The undesirable movement (e.g., wiggle) of the distal end of the prior art devices around fragile body parts (e.g., thin aorta wall, bowels, etc.) may cause unintended lesions, rupture and damage of those sensitive areas. Therefore, the linkage system 120 allows for preferred placement of the electrode actuators 132, 134 in the device 100, which in turn provides comparatively greater stability of the device 100 (e.g., stability of the jaws) during use, when the electrode actuators 132, 134 are actuated by the user. Additionally, the position of the electrode actuators 132, 134 (e.g., symmetrically disposed from the X axis of the device 100) is ergonomically suitable for right-handed or left-handed users.

In the disclosed embodiments, the device 100 does not have a jaw closure lock. A jaw closure lock is understood by those skilled in the art to include a ratchet or other type of mechanism that keeps the jaws in a closed or approximated position after the user closes and releases a handle. Often, to unlock the jaw closure lock, the user must perform another manipulation of the device, such as ‘clicking’ the handle to unlock the jaw closure lock. The omission of a jaw closure lock in the disclosed embodiments reduces the amount of unintentional movement of the device 100 during surgery. The omission of a jaw closure lock in this design also allows the user to quickly and repeatedly dissect, grip, seal, and divide tissue without changing tools and/or without introducing unintentional movement of the device 100 and/or tissue.

In an alternative embodiment, the linkage system 120 may be configured to include a jaw closure lock in the approximate position. For example, when the pivot points 410, 430 and 450 are collinear with respect to each other while the pivot points 420, 440 and 460 are collinear with respect to each other (not shown). Therefore, the device 100 (e.g., first and second floating links 430 and 440) may be configured to lock the jaws in a closed and/or approximate position when the first shank (112) and the first lever link (124) for an angle of approximately 180 degrees therebetween.

In the disclosed embodiments, the linkage system 120 (combined with the increasing force of the load-limiting spring 158 as the jaws 114, 116 approach their approximated position) is configured for the user to experience a lower force to hold the shanks 112, 108 (and hence the jaws 114, 116) in the approximated position. That is, the forces experienced in the gripping mechanism 162 drop at the end of the approximating motion, thus eliminating the need for a jaw closure lock, improving user control and feedback, and reducing user fatigue (by reducing the force needed to hold the jaws closed during tissue sealing). The linkage system 120 is configured to reduce pushback on the user's hand when the jaws 114, 116 are in the approximated position; compared to the pushback experienced by the user's hand just before reaching the approximated position. The reduced pushback on the user's hand is achieved by having 7-bars in the linkage system 120, as described above. The 7-bars linkage system 120 is configured to provide a mechanical advantage when the device 100 reaches the approximate position, such that the compressive force exerted by the user on finger grips 106,110 is less than the force needed to reach the approximate position; therefore, minimizing fatigue of the user's hand in the approximate position.

To improve user feedback, such as to indicate the jaws 114, 116 are in the approximated position, an audible and/or tactile feedback may be provided, such as a click board 174 (FIG. 12). The click board 174 is configured to produce an audible clicking sound to indicate the jaws 114, 116 are in the approximated position or closed.

It should be appreciated that the approximated position of the jaws 114, 116 may be variable. For example, when tissue 200 is disposed, grasped, held, and/or restrained between the jaws 114, 116, the tissue 200 prevents the jaws 114, 116 from contacting each other, although the jaws 114, 116 are disposed in the approximated position or closed (FIG. 13 and FIG. 14). Further examples are: a) when there is no tissue 200 disposed between the jaws 114, 116, or b) very thin tissue 200 is disposed between the jaws 114, 116, yet the jaws 114, 116 are disposed in a closed or approximated position (not shown). The jaws 114 and 116 comprise one or more non-conductive stop members 115 preventing direct contact of the surfaces of the jaws 114 and 116, avoiding short outs, in a manner known to those skilled in the art, yet the jaws 114, 116 are disposed in a closed or approximated position, as shown in FIG. 14.

In some embodiments, the device 100 may be configured to apply pressure on the tissue grasped, held, and/or restrained between the jaws 114, 116 when the jaws 114, 116 are in the approximated position. In some embodiments, the pressure is between about 100 pounds per square inch (about 689 kilopascals) and about 120 pounds per square inch (about 827 kilopascals). In some embodiments, the pressure is between about 50 pounds per square inch (about 345 kilopascals) and about 180 pounds per square inch (about 1,241 kilopascals).

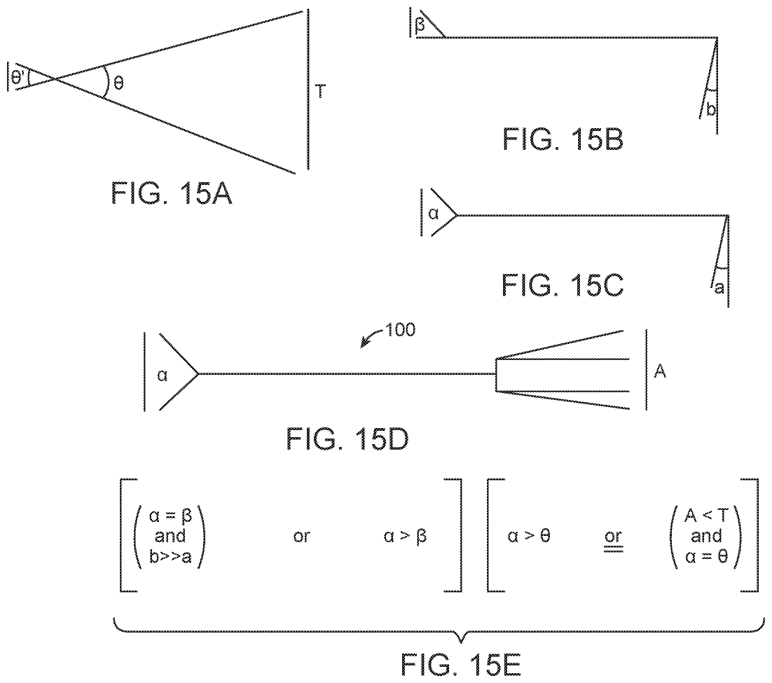

FIGS. 15A-15E illustrate schematics of jaw movements between currently-available prior art devices and the device 100 disclosed according to embodiments of the invention. FIG. 15A depicts schematic of the handles opening angle θ of the prior art hemostat style sealer-divider of FIG. 1. For the device of FIG. 1, the jaw opening angle θ′ is identical or substantially the same as the handle opening angle θ. In order to achieve a wide jaw opening, such as to dissect tissue, the user must open the handle very wide, resulting in a distance T between the handles. The 1:1 correlation of the opening angles (8:8′) is problematic for users with small hands. Additionally, visibility is impeded when working in tight spaces with the device of FIG. 1. FIGS. 15B-15C illustrate schematics of the open jaw angles of the prior art sealer-divider having a pistol grip (FIG. 2). The user of the device of FIG. 2 may have more freedom to open the jaws to a wider angle (e.g., angle α or angle β) compared to the angle θ′ of the device of FIG. 1. The angle α and angle β of the jaws are achieved with less movement on the pistol grip (angles a and b, respectively) of the device of FIG. 2. However, the pistol grip introduces instability, particularly when used in open surgical procedures. The sealer-divider having a pistol grip also typically require a jaw closure lock, which introduces further instability of the device during unlocking.

FIG. 15D is a schematic diagram of the open jaws of the device 100, according to embodiments of the invention. The device 100 overcomes the previously described challenges of the prior art devices. As shown in FIG. 15D, the jaws of the schematic device 100 open to a wide angle α, which is comparable to the angle α (FIG. 15C) in a pistol grip style device, but with a much smaller distance A between handles than the distance T (FIG. 15A) in the hemostat device. In other words, the distance A between handles in the device 100 is significantly less than the distance T between handles of the hemostat device when the jaw opening angle α in the device 100 is equal to the jaw opening angle θ of the hemostat device. Therefore, the device 100 disclosed herein is configured to provide a higher stability than the hemostat device, and greater visibility and a large jaw opening than the pistol grip device. FIG. 15E is a schematic comparison between the handles distance or distal grip angles with their respective jaw opening angles of FIG. 15A-15D

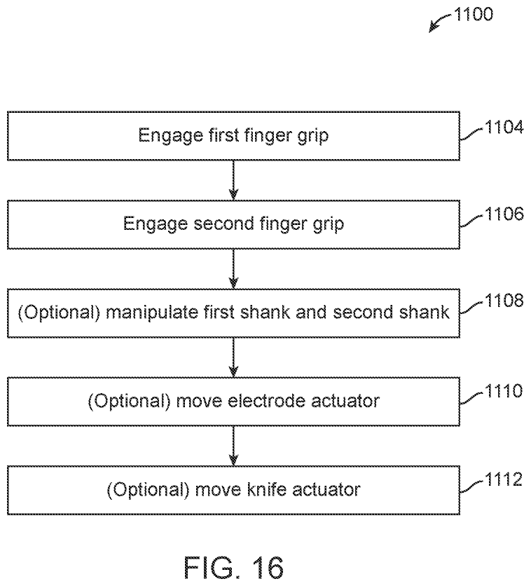

FIG. 16 illustrates a method 1100 for using a sealer-divider-dissector, according to the embodiments of the invention. The device 100 is configured to be used in the manner, actions, and/or steps described by method 1100. The method 1100 may include engaging 1104 the first finger grip with a user's thumb on a first hand. The method 1100 may include engaging 1106 the second finger grip with at least one of the user's middle finger, ring finger, or little finger on the first hand.

Optionally, the method 1100 may include manipulating 1108 the first shank and the second shank. Manipulating 1108 may include, using the user's thumb, manipulating the first shank, and, using the at least one of the user's middle finger, ring finger or little finger on the same hand, and manipulating the second shank, causing the pair of jaws to move between the open position and the closed or approximated position. Manipulating 1108 the first and second shanks may include, using the user's thumb and the at least one of the ring finger or little finger on the first hand, applying an opening motion to the first shank and the second shank, moving the pair of jaws toward the open position, and using the user's thumb and the at least one of the ring finger or little finger on the first hand, applying a approximating motion to the first shank and the second shank, moving the pair of jaws toward the approximated position.

Optionally, the method 1100 may include, using at least one of the user's pointer finger, middle finger, or ring finger on the first hand, to move 1110 the electrode actuator (e.g., proximally) to activate the first and second electrodes.

Optionally, the method 1100 may include, using at least one of the user's pointer finger or middle finger on the first hand, to move 1112 the knife actuator (e.g., proximally) to effectuate a distal movement of the knife.

Additionally, the method 1100 may include moving the electrode actuator (e.g., proximally) parallel to an axis defined by the elongated shaft 118 using the user's middle finger on the user's first hand and moving the knife actuator (e.g., proximally) using the user's pointer finger on the user's first hand.

Additionally, the method 1100 may include disengaging the load-limiting spring when the pair of jaws approaches the open position and engaging the load-limiting spring when the pair of jaws are in the closed or approximated position.

Additionally, the method 1100 may include moving the pair of jaws between the open position and the closed or approximated position a plurality of times without engaging a jaw closure lock.

Although particular embodiments have been shown and described herein, it will be understood by those skilled in the art that they are not intended to limit the present inventions, and it will be obvious to those skilled in the art that various changes, permutations, and modifications may be made (e.g., the dimensions of various parts, combinations of parts) without departing from the scope of the disclosed inventions, which is to be defined only by the following claims and their equivalents. The specification and drawings are, accordingly, to be regarded in an illustrative rather than restrictive sense. The various embodiments shown and described herein are intended to cover alternatives, modifications, and equivalents of the disclosed inventions, which may be included within the scope of the appended claims.

1. A surgical device, comprising:
a distal portion having a pair of jaws configured to move between an open position and an approximated position for manipulating tissue disposed therebetween;
a proximal portion having a housing and a hemostat style gripping mechanism, the hemostat style gripping mechanism including a first finger grip and a second finger grip;
an elongated shaft positioned between the proximal portion and the distal portion, the elongated shaft defining a longitudinal axis; and
a pull tube at least partially disposed within the elongated shaft,
wherein the hemostat style gripping mechanism comprises a linkage system configured for effectuating movement of the pair of jaws between the open position and the approximated position, the linkage system comprising a first shank having distal end rotatably coupled to the housing at a first fixed pivot point and a proximal end coupled to the first finger grip, a second shank having a distal end rotatably coupled to the housing at a second fixed pivot point and a proximal end coupled to the second finger grip, and a slider link operatively coupled to the first shank and the second shank,
wherein the linkage system is a 7-bar linkage system comprising the first shank, second shank, housing, and slider link, the linkage system further comprising a first lever link rotatably coupled to the first shank at each of a first floating pivot point and the slider link, a second lever link rotatably coupled to the second shank at a second floating pivot point and the slider link, and a slide track link affixed to or defined by the housing and configured to limit the slider link to longitudinal movement relative to the housing, and
wherein the pull tube comprises a proximal end coupled to the slider link and a distal end coupled to the pair of jaws, the pull tube configured to move between a first position and a second position proximal of the first position in response to manipulation of the first shank and/or the second shank, whereby the pair of jaws are moved between the open position and the approximated position.

2. The surgical device of claim 1, wherein the first and second shanks, the first and second lever links, and the first and second floating links are collectively configured to vary a mechanical advantage between the open position and the approximated position.

3. The surgical device of claim 2, wherein when the first and second shanks move from the open position to the approximated position during a stroke, the pair of jaws travel a greater distance during a first half of the stroke than during a second half of the stroke.

4. The surgical device of claim 2, wherein a compressive force needed to move the first and second finger grips closer together decreases as the pair of jaws approaches the approximated position.

5. The surgical device of claim 1, wherein the first and second floating pivot points are configured move outwardly from the longitudinal axis defined by the elongated shaft when the pull tube moves distally.

6. The surgical device of claim 1, wherein the first and second floating pivot points are configured move inwardly from the longitudinal axis defined by the elongated shaft when the pull tube moves proximally.

7. The surgical device of claim 1, wherein the first and second floating pivot points are configured to lock the pair of jaws in the approximated position when the first shank and the first lever link form an angle of approximately 180 degrees therebetween.

8. The surgical device of claim 1, wherein the slider link comprises a spring housing that houses a load-limiting spring, and wherein the load-limiting spring is configured to limit a pull force on the pull tube when the pair of jaws are in the approximated position.

9. The surgical device of claim 8, wherein the load-limiting spring is configured to disengage from the pull tube during a transition of the pair of jaws from the approximated position to the open position.

10. The surgical device of claim 1, wherein the linkage system is configured to prevent locking of the pair of jaws in the approximated position.

11. The surgical device of claim 1, further comprising a tissue sealing system having an electrode actuator, a first electrode disposed on a first jaw of the pair of jaws, and a second electrode disposed on a second jaw of the pair of jaws, wherein the first and second electrodes are configured to seal the tissue disposed between the pair of jaws in response to a proximal movement of either one of the electrode actuators, wherein the electrode actuators move in response to a force applied to either one of the electrode actuators in a direction parallel to the longitudinal axis defined by the elongated shaft.

12. The surgical device of claim 11, wherein the electrode actuators are disposed along and laterally offset from the longitudinal axis defined by the elongated shaft.

13. The surgical device of claim 1, wherein the linkage system is configured to vary a mechanical advantage between the open position and the approximated position.

14. The surgical device of claim 13, wherein when the first and second shanks of the linkage system move from the open position to the approximated position during a stroke, the pair of jaws travel a greater distance during a first half of the stroke than during a second half of the stroke, and wherein the mechanical advantage decreases throughout the stroke.

15. The surgical device of claim 13, wherein a compressive force needed to move the first and second finger grips decreases as the pair of jaws approaches the approximated position.

16. The surgical device of claim 1, further comprising a protrusion coupled to the slider link, wherein at least a portion of the protrusion is slidably disposed within a guide member, and wherein the protrusion and the guide member are configured to prevent rotation of the slider link.

17. A surgical device configured to manipulate tissue, the surgical device comprising:
a handle operably coupled to an end effector by a linkage system and an elongated shaft, the handle comprising a first shank and a second shank, the end effector comprising opposing jaws selectively movable between an open position and an approximated position when the handle is actuated,
wherein the linkage system is configured so that a stroke of the handle moves the opposing jaws to an approximated position to engage tissue disposed therebetween by moving the first and second shanks closer together, and wherein a mechanical advantage varies during the stroke whereby the opposing jaws travel a greater distance during a first half of the stroke than during a second half of the stroke.

18. The surgical device of claim 17, wherein the linkage system is configured such that moving the first and second shanks causes the first shank to rotate about a first fixed pivot of the linkage system and causes the second shank to rotate about a second fixed pivot the linkage system, and wherein the first and second fixed pivots are laterally offset from a longitudinal axis defined by the elongated shaft.

19. The surgical device of claim 17, further comprises one or more electrode actuators each having a contact surface for a user's finger, wherein at least one of the electrode actuators is disposed along and laterally offset from the longitudinal axis defined by the elongated shaft.

20. The surgical device of claim 19, wherein the contact surface of the at least one of the actuators is radially offset from the longitudinal axis by a first distance, and the fixed pivot points are radially offset from the longitudinal axis by a second distance greater than the first distance.
